(19) United States
(12) Patent Application Publication
(10) Pub. No.: US 2025/0268498 A1
(43) Pub. Date: Aug. 28, 2025

(54) METHODS AND SYSTEMS FOR DETERMINING STRESS RESILIENCY

(71) Applicant: Sequelae, Inc., Minneapolis, MN (US)

(72) Inventors: Steven J. Catani, Athens, GA (US); Benjamin C. Wiegand, Yardley, PA (US); Douglas A. Granger, Coto de caza, CA (US); James La Belle, Encinitas, CA (US)

(73) Assignee: Sequelae, Inc., Minneapolis, MN (US)

(21) Appl. No.: 19/065,033

(22) Filed: Feb. 27, 2025

Related U.S. Application Data
(60) Provisional application No. 63/558,333, filed on Feb. 27, 2024.

(51) Int. Cl. A61B 5/16; A61B 10/00

(52) U.S. Cl. CPC A61B 5/165; A61B 10/0051

(57) ABSTRACT

To measure stress resiliency in an individual, a first stress indicator level is measured before an activity. Then, at least a second stress level indicator and a third stress level indicator are measured at two or more time points (e.g., immediately following the activity and after a cool-down time). The stress resiliency can then be calculated as a function of the first stress indicator level, the second stress indicator level, and the third stress indicator level at the different times.

### CROSS-REFERENCE TO RELATED APPLICATIONS

This application claims the benefit of U.S. Application No. 63/558,333, filed on Feb. 27, 2024, which is incorporated herein by reference in its entirety.

### BACKGROUND

Various embodiments relate generally to healthcare systems, methods, devices and computer programs and, more specifically, relate to determining stress resiliency of an individual member of a population.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Social media, where individuals can interact with others online, connect many individuals within a community. The online community can be utilized to help build positive behaviors and encourage people to make improvements in their lives. Additionally, computers have enabled new ways to test people for various conditions.

The traditional strategy for operationalizing the measurement of stress involves biobehavioral assessment, including self-reporting, observation, and biological sampling. These measures involve self-report/recall of features and frequency of specific events and subjective experiences associated with the nature and duration of affective states of individuals. They may also include the content of reflections, posts, or derivatives of metrics associated with patterns of engagement in the community on-line network.

On the biological side, two main components of the psychobiology of the stress response are involved, activity of the hypothalamic-pituitary-adrenal (HPA) axis, and the sympathetic branch of the autonomic nervous system (SNS). Reactivity and regulation of these systems can be measured using salivary cortisol (HPA) and either salivary alpha-amylase or heart rate variability (SNS).

The subjective experience of a stressor, rather than the objective features of the event(s) themselves, is central to understanding individual differences in a stressor's biological consequences and therefore, both behavioral and biological measures are important when measuring stress. The association between stress-related behavior and biology is also very dependent on the social context. Measurements taken in the context of everyday life or in response to standardized “challenge” tasks designed to represent key features of those experiences have a high value (e.g., in contrast to “labs” obtained using single measures collected during clinic visits). Here, “high value” refers to the idea that a variation in these parameters when measured in ecologically valid settings are more likely to predict individual differences in the health and social outcomes of primary interest.

What is needed is a way to build upon the social media and access that computers have in order to build healthy lifestyles and develop healthy behaviors as well as improving techniques to measure stress.

### SUMMARY

Example aspects of the present disclosure include:

A method to determine stress resiliency in an individual according to at least one embodiment of the present disclosure comprises determining the stress resiliency by: receiving a first stress indicator level measured prior to an activity; receiving at least a second stress indicator level and a third stress indicator level measured at two or more time points after the activity, wherein the activity is operable to induce a stress response; and determining the stress resiliency as a function of the first measured stress indicator level, the second measured stress indicator level, and the third measured stress indicator level, wherein the first stress indicator level, the second stress indicator level, and the third stress indicator level form a plurality of stress indicator levels; determining a recommendation for an intervention based on the stress resiliency; providing the recommendation to a user; determining a change in the stress resiliency over a plurality of iterations of determining the stress resiliency; and determining an efficacy of the intervention based on the change in the stress resiliency over the plurality of iterations.

Any of the aspects herein, wherein the plurality of stress indicator levels is a cortisol level.

Any of the aspects herein, wherein the plurality of stress indicator levels is calculated as an index of the levels of several stress indicators.

Any of the aspects herein, wherein the plurality of stress indicator levels is measured using saliva.

Any of the aspects herein, wherein the activity is a game that adapts to a user playing the game to provide a constant degree of difficulty.

Any of the aspects herein, wherein the activity induces a mental challenge or psychological challenge.

Any of the aspects herein, wherein the stress resiliency is calculated as a function of the difference between the first stress indicator level and both the second stress indicator level and the third indicator level.

Any of the aspects herein, wherein the stress resiliency is determined as a function of the time taken for a fourth stress indicator level taken after the third stress indicator level to return to the range of the first stress indicator level.

Any of the aspects herein, wherein the stress resiliency is determined as a function of a slope of the second stress indicator level and the third stress indicator level.

Any of the aspects herein, wherein the stress resiliency is calculated as an index of a function of the difference between the first stress indicator level and both the second stress indicator level and the third stress indicator level, the time it takes for a fourth stress indicator level taken after the third stress indicator level to return to the range of the first stress indicator level, and a function of a slope of both the second stress indicator level and the third stress indicator level.

Any of the aspects herein, wherein measuring the plurality of stress indicator levels comprises using a sample kit to collect at least one of: stool, blood, and saliva.

Any of the aspects herein, wherein measuring the plurality of stress indicator levels comprises taking a reading using at least one of: a psychometric instrument, a heart rate monitor, and a pulse oximeter.

Any of the aspects herein, wherein measuring the plurality of stress indicator levels comprises sharing the plurality of stress indicator levels using a digital communication network.

A method to determine stress resiliency in an individual according to at least one embodiment of the present disclosure comprises determining the stress resiliency by: receiving a first stress indicator level measured prior to an activity; receiving at least a second stress indicator level and a third stress indicator level measured at two or more time points after the activity, wherein the activity is operable to induce a stress response; and determining the stress resiliency as a function of the first measured stress indicator level, the second measured stress indicator level, and the third measured stress indicator level, wherein the first stress indicator level, the second stress indicator level, and the third stress indicator level form a plurality of stress indicator levels; determining a recommendation for an intervention based on the stress resiliency; and providing the recommendation to a user.

Any of the aspects herein, wherein the method is performed at multiple instances, and wherein the method further comprising determining a change in the stress resiliency over multiple iterations of the method.

Any of the aspects herein, further comprising determining an efficacy of an intervention based on the change in the stress resiliency over the multiple instances.

Any of the aspects herein, wherein the stress resiliency is calculated as a function of the difference between the first stress indicator level and both the second stress indicator level and the third indicator level.

Any of the aspects herein, wherein the stress resiliency is determined as a function of the time taken for a fourth stress indicator level taken after the third stress indicator level to return to the range of the first stress indicator level.

Any of the aspects herein, wherein the stress resiliency is determined as a function of a slope of the second stress indicator level and the third stress indicator level.

A system according to at least one embodiment of the present disclosure comprises a computer processor; a data repository in communication with the computer processor and storing: individual statistics having a stress resiliency and historic readings, stress indicator data having at least a first stress indicator level, a second stress indicator level, a third stress indicator level and reading timing data, an activity, and a recommendation; an activity controller which, when executed by the computer processor, administers the activity; a stress resilience generator which, when executed by the computer processor, determines the stress resiliency; a digital communications network which, when executed by the computer processor, provides a network for members of a population to interact with each other and for an individual member to access a stress resiliency test for determining the individual member's stress resiliency; a server controller which, when executed by the computer processor: determining the stress resiliency by: receives the first stress indicator level prior to the activity; receives at least the second stress indicator level and the third stress indicator level at two or more time points after the activity; and determines a stress resiliency as a function of the first stress indicator level, the second stress indicator level, and the third stress indicator level; determines a recommendation for an individual based on the stress resiliency; provides the recommendation to the user; determines a change in the stress resiliency over a plurality of iterations of determining the stress resiliency; and determines an efficacy of the intervention based on the change in the stress resiliency over the plurality of iterations.

### BRIEF DESCRIPTION OF THE DRAWINGS

Aspects of the described embodiments are more evident in the following description, when read in conjunction with the attached Figures.

FIG. 1 shows a simplified diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Stress resiliency or stress resilience refers to the ability for a person to adapt well and recover quickly from stress and is referred to as “adaptive calibration.” People who have a high level of stress resilience can adaptively calibrate their responses to stress. These people may be more able to adapt to daily stressors without getting overwhelmed. Further, such people can cope well with major stressful life events, are resilient after difficult experiences, and can return to a baseline level of functioning after such experiences. Such level of stress resiliency can help maintain a positive mindset and attitude despite challenges.

Stress can create many problems for those who have a low level of stress resiliency as there are differences between the short-term effects of acute stress and the long-term effects of chronic stress. In the short run, stress is considered part of everyday life. Behavioral, psychological, and biological changes induced by acute stress are largely considered adaptive. Everyday stress encodes experiences in our memory, enable efficient and effective use of energy, and link our prior experiences to the present challenges so, we can employ that experience to adapt and adjust.

The consequences of stress become a concern if an individual is regularly activating these stress responsive systems and/or if those responses do not attenuate or habituate over time after accumulating experience with that stressor. In these scenarios, the consistent/persistent activation of these biological systems can, over time, result in wear and tear on downstream biological processes and related biological systems, a phenomenon called “allostatic load.” Individual differences in reactivity and regulation of acute stress, and the allostatic load related to the effects on chronic stress are associated with a wide range of negative mental and physical health consequences.

Prolonged stress can lead to elevated levels of the stress hormone cortisol, which can cause negative effects on a person. For example, elevated levels of cortisol are linked to the development of mood disorders like anxiety and depression. Stress triggers can also cause changes in the brain chemistry that can cause headaches, including tension headaches and migraines. As such, headaches are a very common symptom of chronic stress. High blood pressure is another possible result of stress. Stress activates the sympathetic nervous system, leading to constriction of blood vessels and a rise in blood pressure over time. Chronic stress is a known risk factor for hypertension. Additionally, stress may cause digestive issues as stress can irritate inflammatory bowel diseases like Crohn's disease or ulcerative colitis. It can also lead to diarrhea, constipation, acid reflux, heartburn, and stomach pain as the gut-brain connection is sensitive to stress.

Further, chronic systemic inflammation (CSI) may also be impacted by stress. CSI may be an underlying cause of many diseases and can increase healthcare costs over time if untreated. Such diseases include rheumatoid arthritis (RA) and others. Weakened immune systems are often a result of stress. Chronic activation of the stress response diverts crucial resources away from the functioning of the immune system, making stressed individuals more susceptible to colds, flu, and infections. Further, the stress hormone cortisol is regulated on a 24-hour cycle and can interfere with normal sleep-wake cycles if it stays elevated at night. This may lead to insomnia and poor sleep quality. Stress can also cause weight gain or loss. Some people lose their appetite when stressed (and may lose weight), while others crave unhealthy comfort foods and tend to gain weight during times of chronic stress.

The long-term physical effects underscore the importance of managing stress effectively by whatever healthy coping strategies work for each individual. Unmanaged stress takes a cumulative toll on physical and mental health.

Thus, a high level of stress resiliency is desirable for individuals. Stress resiliency includes behaviors, thoughts, and actions that can be learned and developed. Some key factors associated with stress resilience and the ability to cope with stress include:
Having strong social support and relationships;
Remaining positive, optimistic, and confident when faced with adversity;
Accepting that change is part of living and looking for opportunities to grow;
Maintaining perspective by viewing experiences as temporary and as opportunities to learn;
Practicing good self-care through rest, relaxation, healthy eating, exercise, and attending to emotional needs; and
Learning coping strategies like problem-solving skills, managing emotions, managing expectations.

Engaging in stress management techniques can help increase the level of stress resiliency over time. Such techniques include, for example, meditation, yoga, deep breathing, spending time outdoors, exercising, practicing gratitude, and finding purpose and meaning in life experiences. Having a higher level of stress resiliency does not mean a person will not experience stress and emotional pain, but rather enables a person to manage it well.

Another way to increase the level of stress resiliency includes engaging in online communities. Online communities can also help people learn about healthy lifestyle practices that can improve one's level of stress resiliency. To help incentivize healthy behaviors, members of a population can use tools like in-home tests and biosensors that measure how well their actions to reduce stress or increase stress resiliency are working. The population may be, for example, a population of patients of a healthcare system.

Further, this online program gives access to information and guidance to help members discover the healthy actions that may work best for them and to help with increasing a level of stress resiliency. They can also find a community of members and others who can support them and who they can support through online or arranged in-person conversations. Using their personal computers or devices (such as a cell phone), members can have access to activity trackers, health testing, etc. as well as other tools aimed at improving their health. Such information may be gathered from connected devices, such as, glucose measurements from a continuous glucose monitor, and wearables like Fitbit. Members such as patients and their doctors can also communicate to address issues like food, transportation, and other factors that impact their health and stress resiliency.

Thus, various embodiments provide means for members of a population such as providers and patients to improve their health and stress resiliency by improving lifestyle behaviors. In some instances, this is done through an online portal that helps people find, pass along, and rely on the tools and information that make it easiest to adopt the healthy actions that work best.

In many situations, members can use their personal devices, such as a computer, tablet, cell phone, etc., to interact with a host system on a server. The host system can provide a platform for relaying communications, such as public posts, and/or direct messages, etc., Additionally, the host system can store patient/user information, for example, biometric information, test results, personal data, etc. In some embodiments, the host system may provide services/apps, e.g., monitoring or even games.

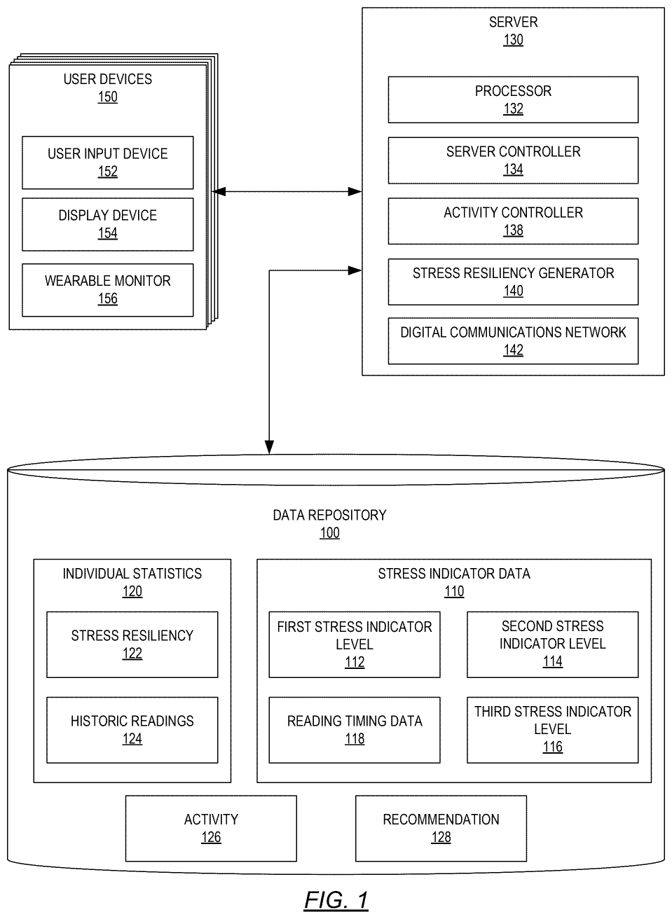

Attention is now turned to the figures. FIG. 1 shows a computing system, in accordance with one or more embodiments. The computing system can be used, for example, to support a digital communication network (DCN) that will enable members of a population to communicate with each other. The computing system can also be used to determine a stress resiliency of an individual member in the population through various testing, determine a recommendation to the individual member based on their stress resiliency, determine a change in the stress resiliency, and determine an efficacy of the recommendation based on the change in the stress resiliency.

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores stress indicator data (110). The stress indicator data (110) includes readings indicative of an individual's stress levels, such as cortisol. The stress indicator data (110) may incorporate multiple readings taken over various times. For example, a first stress indicator level (112) may be measured prior to an activity (126) (described below). Further, a second stress indicator level (114) and a third stress indicator level (116) may be measured after the activity (126). In some embodiments, the stress indicator data (110) may include more or less stress indicator levels. For example, the stress indicator data (110) may include a fourth stress indicator level, a fifth stress indicator level, etc. The stress indicator data (110) also includes reading timing data (118), which provides information regarding when various stress indicator levels are taken such as the date and/or time a stress indicator level is taken.

The stress indicator data (110) may be measured using, for example, blood or a saliva sample that can be taken at-home or at a clinic. The stress indicator data (110) may also be measured or based on user feedback provided to, for example, the DCN (142) via a user device (150). An individual body's physiological response to psychological, or mental stress can also be used to measure the stress indicator data (110). Such response can also be used to look at chronic systemic inflammation, such as to detect a prodromal signal.

The data repository (100) also stores individual statistics (120). The individual statistics (120) may include personal information of an individual that can be used in determining their stress resiliency (122) (described below), such as, height, weight, etc. This data can also include one or more previously calculated stress resiliency (122) and historic readings (124) of stress resiliency (122).

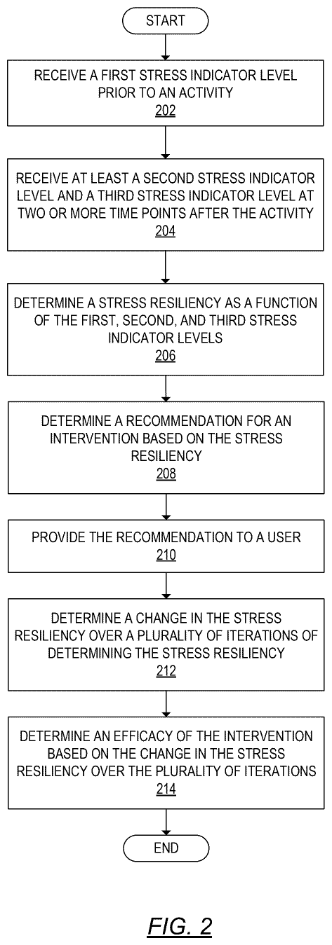

The data repository (100) also stores the stress resiliency (122). The stress resiliency (122) is a member's ability to adapt and/or recover from stress. The stress resiliency (122) is determined or generated based on the stress indicator data (110) and/or the individual statistics (120). The stress resiliency (122) can be measured by using various activities, which will be described in detail in FIG. 2.

The data repository (100) also stores the activity (126). The activity (126) may be, for example, various digital stimulus or challenges, such as an adaptive game, a controlled encounter with another individual, therapeutic event, or an audial and/or visual stimulus (e.g., a movie, a piece of music or art). The activity (126) may also be a physical activity such as, for example, running, climbing, or swimming. In such examples, the activity (126) may include instructions or prompts for completing the physical activity. The activity (126) may also be used to decrease stress in some embodiments.

In some embodiments, the activity (126) may be a game programmed to adapt to provide a constant degree of difficulty so as to induce a mental challenge and/or a psychological challenge. By linking the stress indicator level measurement to an activity (126) that is known to have an impact on the stress indicator level measured, e.g., measuring stress after an activity that is known to increase stress, information about the sensitivity of the individual's response to the activity (126) and the time to recover from the activity (126) can be obtained. Since the activity (126) is standardized (at least for the individual), it allows a new range of stress indicator level measurements to be used to encourage and measure the effects of behavior change. It also allows smaller changes to be seen, which is often important in initiating behavior changes to increase stress resiliency.

The data repository (100) also stores a recommendation (128). The recommendation (128) is generated for the individual member based on their stress resiliency (122) or a change in their stress resiliency (122). For example, if the level of the individual member's stress resiliency is low, indicating a low ability to manage stress, then the recommendation (128) may provide suggestions to increase the individual member's stress resiliency (122). Such suggestions may include, for example, interacting with other members in a digital communication network (DCN) to learn how other members manage their stress, yoga, meditation, attending a seminar on reducing stress and increasing stress resiliency, or medical intervention. In some instances, the recommendation (128) may be provided to the individual member's healthcare provider. In some embodiments, if the level of the individual member's stress resiliency (122) is high, the recommendation (128) may be to encourage the individual member to share their techniques for increasing and maintaining a high level of stress resiliency (122).

In some embodiments, the recommendation (128) may be provided to the population or sub-groups of the population. For example, a sub-group of members may have similar levels of stress resiliency (122) and the recommendation (128) for suggestions on how to increase their levels of stress resiliency (122) may be provided to the sub-group.

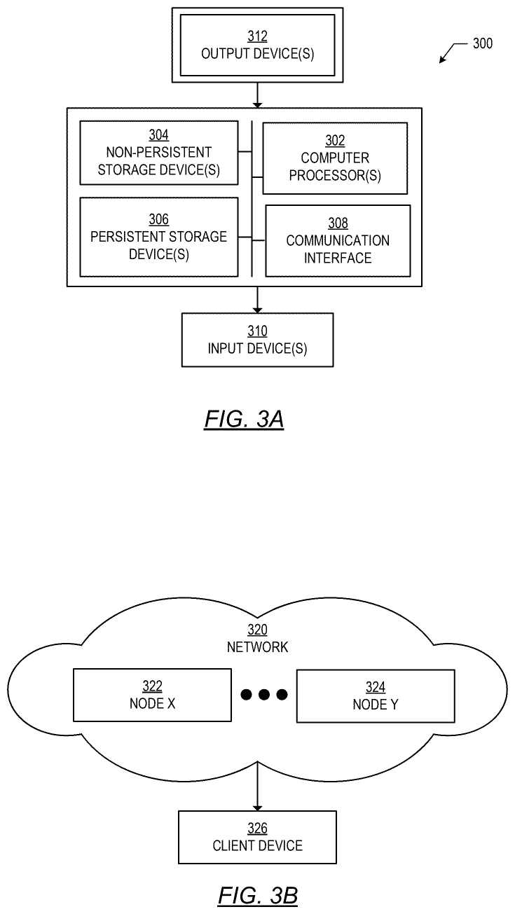

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as an activity controller (138) or a stress resiliency generator (140). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the activity controller (138) or the stress resiliency generator (140). An example of the computer processor (132) is described with respect to the computer processor(s) (302) of FIG. 3A.

The server (130) also may include a server controller (134). The server controller (134) is software or application specific hardware which, when executed by the computer processor (326), controls and coordinates operation of the software or application specific hardware described herein. Thus, the server controller (134) may control and coordinate execution of the activity controller (138), and the stress resiliency generator (140).

The server (130) also includes the activity controller (138). The activity controller (138) is software or application specific hardware which, when executed by the computer processor (132) provides the activity as a digital stimulus or challenge to the user in order to generate a stress response, such as through a game. In embodiments where the activity is a physical activity, the activity controller (138) may provide prompts to the individual to perform the activity.

The server (130) also includes a stress resiliency generator (140). The stress resiliency generator (140) is software or application specific hardware which, when executed by the computer processor (132), performs the method of FIG. 2. The stress resiliency generator (140) receives, as input, the stress indicator data (110) and determines the stress resiliency (122). In some embodiments, the stress resiliency generator (140) may also receive individual statistics (120) which are used in the generation of the stress resiliency (122).

The server (130) also includes the digital communications network (DCN) (142). The DCN (142) is a network through which members of a population can interact with each other, or with a system supported by the DCN (142). For example, an individual member can enroll for a stress resiliency testing to determine their stress resiliency (122) through the DCN (142). In another example, the individual member can interact with other members to learn about techniques for increasing their level of stress resiliency (122).

In some embodiments, the DCN (142) can administer the activity to determine an individual member's stress resiliency (122). For example, if the activity (126) is a computer game, the DCN (142) can provide the computer game to an individual member. The DCN (142) can also be used to receive input from the individual member such as measurements, feedback, or comments regarding the activity. For example, the individual member may use an application on their mobile device to send in the feedback or comments to the DCN (142), or may wear a wearable device or monitor (156) that can measure and send the measurements to the DCN (142).

The DCN (142) can also provide means for members of the population to communicate with each other. Members can share results from their stress resiliency testing or assessment and can also share techniques for improving their stress resiliency (122). The DCN (142) can also be used to deliver or transmit the recommendation (128) to the individual member based on their stress resiliency (122).

In the DCN (142), patient messages may be ordered to prioritize messages from members whose level of stress resiliency (122) is higher over time. These communications between members of the population can be one-to-one, one-to-many, one-to-system, system-to-one, or system-to-many. The system may also feature an AI bot that derives its communications from analysis of communications in the digital communication network and/or biometrics provided to the DCN (142).

In another, non-limiting embodiment, patient messages in the DCN (142) may be ordered to prioritize messages from members who have had interventions that increase stress resiliency (122). The interventions can be lifestyle behaviors and/or seeking professional care.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send stress indicator data (110) to the server (130). In an alternative embodiment, a separate wearable device or monitor (156) may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to determine a stress resiliency of an individual and, in some instances, generate a recommendation to help the individual increase their level of stress resiliency. Determining the stress resiliency for an individual or groups of people can provide a foundation for managing healthcare risk in a population. By measuring stress resiliency, it may be possible to detect serious medical conditions before they worsen. Thus, measuring the general level of stress resiliency in a population can be used to manage healthcare.

In accordance with an embodiment a method performs an iterative process in which a stress resiliency for an individual is determined. The iterative process can be repeated multiple times or executed at one time.

At Step 202, a first stress indicator level prior to an activity is received. It will be appreciated that in some embodiments, more than one stress indicator level can be received prior to execution of the activity. The first stress indicator level can be received by a digital communications network (DCN) such as the DCN (142) via a user device such as the user device (150). The activity, as previously described, is used to induce a change in the stress indicator level of the individual participating in the activity for the purpose of determining a stress resiliency such as the stress resiliency (122). The activity can be, for example, a normal life activity or food routinely consumed. The activity may be any activity that can be standardized, repeated, and induces a change in the stress indicator level in a known way.

The first stress indicator level can be part of a plurality of stress indicator levels and also part of stress indicator data such as the stress indicator data (110). The plurality of stress indicator levels can be a measurement of a stress hormone such as, for example, cortisol and/or an enzyme, such as, alpha-amylase. The stress indicator can be measured in saliva or capillary blood. In some embodiments, the level of the stress indicator is calculated as an index of the levels of several stress indicators.

The plurality of stress indicator levels can be measured at-home or at a clinic. The plurality of stress indicator levels can be biometrics derived from saliva or blood or breath or stool samples, as well as those derived from heartrate, accelerometer samples, body temperature, skin conductivity, as well as signals derived from wearables. It can also be obtained from psychometric instruments and emergency medical assistant derived data.

At Step 204, at least a second stress indicator level and a third stress indicator level measured at two or more time points after the activity is received. The activity may be administered by, for example, the DCN using an activity controller such as the activity controller (138).

The second stress indicator level and the third stress indicator level can also be received by the DCN via the user device. In some embodiments, the Step 204 may include receiving additional stress indicator levels at different time points after the activity. For example, a fourth stress indicator level may be obtained after the third stress indicator level, a fifth stress indicator level may be obtained after the fourth stress indicator level, etc.

The second stress indicator level, third stress indicator level, fourth stress indicator level, etc. can also be part of the plurality of stress indicator levels, though the second stress indicator level, third stress indicator level, fourth stress indicator level, etc. are measured after the individual has interacted or participated in the activity.

At Step 206, a stress resiliency is determined as a function of the first stress indicator level, the second stress indicator level, and the third stress indicator level. The stress resiliency can be determined by and received as output from a stress resiliency generator such as the stress resiliency generator (140). For example, the stress resiliency can be determined by the stress resiliency generator (140) as a function of the time it takes for the second stress indicator level and the third stress indicator level (e.g., the stress indicator levels after the activity) to return to the range of the first stress indicator level (e.g., the stress indicator level prior to the activity). In other examples, the stress resiliency can be calculated as a function of the slope of the second stress indicator level and the third stress indicator level (or any number of post-activity stress indicator levels).

Alternatively, the stress resiliency can be calculated as an index of a function of the difference between the first stress indicator level and both the second stress indicator level and the third stress indicator level, the time it takes for the second stress indicator level and the third stress indicator level to return to the range of the first stress indicator level, and a function of the slope of the second stress indicator level and the third stress indicator level. In some embodiments, the first stress indicator level is before or pre-activity and the second stress indicator level and the third stress indicator level are after or post-activity. In such embodiments, the stress resiliency can be calculated based on the difference between the pre-activity stress indicator level and the post-activity stress indicator level(s). In still such embodiments, the stress resiliency can be calculated as a time to return to a baseline stress indicator level (which may be, for example, the first stress indicator level) after the activity has been completed. In still other embodiments, the stress resiliency can be calculated as a combination of factors including the difference between the pre-activity stress indicator level and the post-activity stress indicator level and/or the time to return to a baseline stress indicator level after the activity has been completed.

At Step 208, a recommendation for an intervention based on the stress resiliency is determined. As previously described, the recommendation may be determined or generated based on the stress resiliency, as determined in the Step 206. For example, in instances where the stress resiliency is determined to be low, the recommendation may provide suggestions to increase the individual member's stress resiliency. In another example, when the stress resiliency is determined to be high, the recommendation may provide suggestions for the individual member to share their techniques for increasing and maintaining a high level of stress resiliency with other members through the DCN.

At Step 210, the recommendation is provided to a user. The recommendation may be provided to the user from the DCN via the user device. In other embodiments, the recommendation may also be provided to the user from any device or system.

At Step 212, a change in the stress resiliency is determined over a plurality of iterations of determining the stress resiliency (e.g., a plurality of iterations of the steps 202-210). The change in the stress resiliency may simply be a difference between the stress resiliency at different iterations. In other embodiments, the stress resiliency may be averaged over the different iterations.

At Step 214, an efficacy of the intervention is determined based on the change in the stress resiliency over the plurality of iterations. In some embodiments, the efficacy may be determined based on a threshold. For example, if the change in the stress resiliency is below the threshold, then this may indicate that the recommendation provided to the user was ineffective. This may result in adjustments or changes to the recommendation. In another example, if the change in the stress resiliency is above the threshold, then this may indicate that the recommendation provided to the user was effective. In such example, the recommendation may not be changed for the user.

In some embodiments, additional steps can be taken. For example, members of the population can be made aware of a test for a secondary related generalized health marker and/or provided the members with the means to measure their level of a secondary related generalize health marker. The secondary related generalize health marker may be chronic systemic inflammation which can be measured using a blood or saliva sample. This sample can be taken at-home. The chronic systemic inflammation level may be derived from levels in the blood or saliva sample from the measurement of one or more of CRP, IL-2, TNF-alpha, etc. In other situations, the secondary related generalized health marker information can be measured using a wearable device.

In the DCN, the order of messages shown to a member of the social network is derived, in part, from the prioritizing messages from members who have used the secondary related generalized health marker testing. The secondary related generalized health marker may be selected to be related to the members epigenome; the member's stress, anxiety, or depression; the members activity; and/or the members social interaction.

The method of FIG. 2 described above may have more or less steps than shown. Further, steps may be repeated as needed. For example, the Steps 202, 204, 206, 212, and 214 may be repeated to evaluate if the recommendation provided in the Step 208 and 210 was effective.

A specific example of the above method of FIG. 2 will now be described. In such example, the stress indicator levels may be measured using biometrics. As in the case of self-controlled biomarkers, the activities do not need to be the same for each individual, only for the individual being tested and the activities can be adjusted so the measured stress indicator level after the activity is in a range that provides useful information. Over time, the stress indicator levels measured after an activity can be used to adjust the activity to keep the stress indicator levels in a meaningful range. Such adjustments enable the system to look at a stress indicator level result at a point in time instead of at the first stress indicator level of a series of stress indicator levels as new biomarkers. This is due to a change in behavior (as a result of the activity) effecting a change in a biometric is different than evaluating how a change in behavior moves the patient from an abnormal stress indicator level range to a normal stress indicator level range.

In the specific example, the activity may be a video game known to induce stress in an individual. A first biometric can be obtained from the individual prior to starting the video game. Then, a second and a third biometric can be obtained from the individual after the individual has completed the video game at two different time points. A stress resiliency of the individual can then be determined based on the first, second, and third biometrics.

In such example, a level of the stress resiliency may be below a threshold, which indicates that the individual has a low level of stress resiliency. Thus, a recommendation may be generated and provided to the user that suggests the individual to interact with other members of a DCN having the same or similar stress resiliency results. The recommendation may also suggest that the individual share their reflections on their stress resiliency with other members of the DCN. The recommendation to interact with other members of the DCN may be provided to the individual by the DCN via a user device that the individual owns such as a smartphone, laptop, wearable device, etc.

The individual may then have their stress resiliency redetermined by participating in the activity again and having their first, second, and third biometrics remeasured. A change in the individual's stress resiliency indicates that the recommendation was either effective or ineffective. For example, if the individual's stress resiliency increases, then the recommendation may be determined to be effective. If the individual's stress resiliency decreases, then the recommendation may be determined to be ineffective.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN) such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to determine stress resiliency in an individual, the method comprising:
determining the stress resiliency by:
receiving a first stress indicator level measured prior to an activity;
receiving at least a second stress indicator level and a third stress indicator level measured at two or more time points after the activity, wherein the activity is operable to induce a stress response; and
determining the stress resiliency as a function of the first measured stress indicator level, the second measured stress indicator level, and the third measured stress indicator level,
wherein the first stress indicator level, the second stress indicator level, and
the third stress indicator level form a plurality of stress indicator levels;
determining a recommendation for an intervention based on the stress resiliency;
providing the recommendation to a user;
determining a change in the stress resiliency over a plurality of iterations of determining the stress resiliency; and
determining an efficacy of the intervention based on the change in the stress resiliency over the plurality of iterations.

2. The method of claim 1, wherein the plurality of stress indicator levels is a cortisol level.

3. The method of claim 1, wherein the plurality of stress indicator levels is calculated as an index of the levels of several stress indicators.

4. The method of claim 1, wherein the plurality of stress indicator levels is measured using saliva.

5. The method of claim 1, wherein the activity is a game that adapts to a user playing the game to provide a constant degree of difficulty.

6. The method of claim 1, wherein the activity induces a mental challenge or psychological challenge.

7. The method of claim 1, wherein the stress resiliency is calculated as a function of the difference between the first stress indicator level and both the second stress indicator level and the third indicator level.

8. The method of claim 1, wherein the stress resiliency is determined as a function of the time taken for a fourth stress indicator level taken after the third stress indicator level to return to the range of the first stress indicator level.

9. The method of claim 1, wherein the stress resiliency is determined as a function of a slope of the second stress indicator level and the third stress indicator level.

10. The method of claim 1, wherein the stress resiliency is calculated as an index of a function of the difference between the first stress indicator level and both the second stress indicator level and the third stress indicator level, the time it takes for a fourth stress indicator level taken after the third stress indicator level to return to the range of the first stress indicator level, and a function of a slope of both the second stress indicator level and the third stress indicator level.

11. The method of claim 1, wherein measuring the plurality of stress indicator levels comprises using a sample kit to collect at least one of: stool, blood, and saliva.

12. The method of claim 1, wherein measuring the plurality of stress indicator levels comprises taking a reading using at least one of: a psychometric instrument, a heart rate monitor, and a pulse oximeter.

13. The method of claim 1, wherein measuring the plurality of stress indicator levels comprises sharing the plurality of stress indicator levels using a digital communication network.

14. A method to determine stress resiliency in an individual, the method comprising:
determining the stress resiliency by:
receiving a first stress indicator level measured prior to an activity;
receiving at least a second stress indicator level and a third stress indicator level measured at two or more time points after the activity, wherein the activity is operable to induce a stress response; and
determining the stress resiliency as a function of the first measured stress indicator level, the second measured stress indicator level, and the third measured stress indicator level,
wherein the first stress indicator level, the second stress indicator level, and
the third stress indicator level form a plurality of stress indicator levels;
determining a recommendation for an intervention based on the stress resiliency; and
providing the recommendation to a user.

15. The method of claim 14, wherein the method is performed at multiple instances, and
wherein the method further comprising determining a change in the stress resiliency over multiple iterations of the method.

16. The method of claim 15, further comprising determining an efficacy of an intervention based on the change in the stress resiliency over the multiple instances.

17. The method of claim 14, wherein the stress resiliency is calculated as a function of the difference between the first stress indicator level and both the second stress indicator level and the third indicator level.

18. The method of claim 14, wherein the stress resiliency is determined as a function of the time taken for a fourth stress indicator level taken after the third stress indicator level to return to the range of the first stress indicator level.

19. The method of claim 14, wherein the stress resiliency is determined as a function of a slope of the second stress indicator level and the third stress indicator level.

20. A system comprising:
a computer processor;
a data repository in communication with the computer processor and storing:
individual statistics having a stress resiliency and historic readings,
stress indicator data having at least a first stress indicator level, a second stress indicator level, a third stress indicator level and reading timing data,
an activity, and
a recommendation;
an activity controller which, when executed by the computer processor, administers the activity;
a stress resilience generator which, when executed by the computer processor, determines the stress resiliency;
a digital communications network which, when executed by the computer processor, provides a network for members of a population to interact with each other and for an individual member to access a stress resiliency test for determining the individual member's stress resiliency;
a server controller which, when executed by the computer processor:
determining the stress resiliency by:
receives the first stress indicator level prior to the activity;
receives at least the second stress indicator level and the third stress indicator level at two or more time points after the activity; and
determines a stress resiliency as a function of the first stress indicator level, the second stress indicator level, and the third stress indicator level;
determines a recommendation for an individual based on the stress resiliency;
provides the recommendation to the user;
determines a change in the stress resiliency over a plurality of iterations of determining the stress resiliency; and
determines an efficacy of the intervention based on the change in the stress resiliency over the plurality of iterations.
